(19) United States
(12) Patent Application Publication
(10) Pub. No.: US 2025/0082337 A1
(43) Pub. Date: Mar. 13, 2025

(54) Implant Delivery System

(71) Applicant: MicroVention, Inc., Aliso Viejo, CA (US)

(72) Inventors: Heath Bowman, Trabuco Canyon, CA (US); Nga Ting Wong, Alhambra, CA (US); Diana N. Valencia, Hawaiian Gardens, CA (US)

(73) Assignee: MicroVention, Inc., Aliso Viejo, CA (US)

(21) Appl. No.: 18/886,808

(22) Filed: Sep. 16, 2024

Related U.S. Application Data
(62) Division of application No. 16/705,097, filed on Dec. 5, 2019, now Pat. No. 12,114,863.
(60) Provisional application No. 62/775,816, filed on Dec. 5, 2018.

(51) Int. Cl. A61B 17/12; A61B 17/00; A61B 90/00; A61F 2/01; A61F 2/966; A61F 6/22

(52) U.S. Cl. CPC A61B 17/1214; A61B 17/12113; A61F 6/22; A61B 2017/00526; A61B 2017/00632; A61B 2017/12068; A61B 2090/3983; A61F 2/011; A61F 2002/9665

(57) ABSTRACT

An implant delivery system having an implant-pusher junction that has improved flexibility due to a shortened heater and tapered marker band. The heater capsule includes a coil having a complex shape made possible through additive manufacturing. The complex shape generates increased heat over a shorter length when compared to traditional linear coils. The marker band includes a tapered end that allows the implant to pivot relative to the pusher.

### RELATED APPLICATIONS

This application is a continuation of and claims priority to U.S. patent application Ser. No. 16/705,097 filed Dec. 5, 2019 entitled Implant Delivery System, which claims benefit of and priority to U.S. Provisional Application Ser. No. 62/775,816 filed Dec. 5, 2018 entitled Implant Delivery System, both of which are hereby incorporated herein by reference in their entireties.

### FIELD OF THE INVENTION

The present invention relates to systems and methods for delivering implant devices to a target site or location within the body of a patient.

### BACKGROUND OF THE INVENTION

Delivery of implantable therapeutic devices by less invasive means has been demonstrated to be desirable in numerous clinical situations. For example, vascular embolization has been used to control vascular bleeding, to occlude the blood supply to tumors, to occlude fallopian tubes, and to occlude vascular aneurysms, particularly intracranial aneurysms. In recent years, vascular embolization for the treatment of aneurysms has received much attention. Implants used to treat aneurysms are often convoluted or coiled lengths of wound wire and are referred to as “microcoils.” Microcoils work by filling an aneurysm causing the blood flow through the aneurysm to slow or stop, thereby inducing thrombosis within the aneurysm.

Vascular aneurysms can have extremely thin walls and puncturing an aneurysm wall results in a stroke. Because the walls are so delicate, microcoils are extremely flexible and have very little structural integrity. These microcoils are connected to a pusher system to enable delivery to the treatment site. Ideally, the microcoils and pusher would be sufficiently flexible in order to navigate the vasculature and enable placement within the aneurysm region. However, with traditional designs, there can be significant stiffness, particularly at the pusher-implant connection junction at the proximal end of the microcoil implant.

This rigidity of the proximal end of the coil can be partially attributed to the connection the coil has with the pusher, which is typically attached to the implant and is used to drive the pusher out of a delivery catheter. Often, the proximal end of the coil includes a rigid cylindrical marker band that is held against the distal end of the pusher, adding rigidity to the coil. Additionally, a heater element contained within the distal end of the pusher prevents bending of the distal tip of the pusher, resulting in a rigid implant-pusher junction that increases delivery difficulty. The combination of the heater coil and the marker band thus results in a rigid length at the junction of the implant and the device.

Though several coils and delivery systems have been developed, none have specifically addressed the rigidity at the junction of the implant and the pusher that is due to the sizes and shapes of the implant marker band and/or the length of the heater. Examples of these coils and detachment systems for implant devices are described in the prior art including U.S. Pat. No. 5,895,385 to Guglielmi et al. and U.S. Pat. No. 5,108,407 to Geremia et al., the contents of which are hereby incorporated by reference. Examples of systems using a heater to detach an implant device are disclosed in U.S. Pat. No. 9,867,622 to Bowman, U.S. Pat. No. 6,500,149 to Gandhi et al. and U.S. Pat. No. 4,346,712 to Handa et al., the contents of which are hereby incorporated by reference, describe the use of a heater to detach and deploy the implant device. Additionally, U.S. Pat. No. 8,182,506 to Fitz et al., U.S. Patent Pub. No. 2010/0268204 to Teiu et al., and U.S. Patent Pub. No. 2011/0301686 to Bowman et al., also pertain to thermal detachment systems and are hereby incorporated by reference in their entireties.

It would thus be desirable to develop a coil delivery system that maintains flexibility at the junction of the coil and the pusher. Such a device would promote ease of coil and device positioning during delivery as well as the prevention of coil migration out of the aneurysm after release.

### SUMMARY OF THE INVENTION

The present invention pertains to a heater and a marker band designed for use with an implant delivery and detachment system used to position and deploy implantable devices such as coils, stents, filters, and the like within a body cavity including, but not limited to, blood vessels, fallopian tubes, malformations such as fistula and aneurysms, heart defects (e.g. left atrial appendages and sepal openings), and other luminal organs.

The heater designs of the present invention include complex shapes and a double-element design made possible through modern manufacturing techniques. The double-element design shortens the length of the heater necessary to achieve the temperatures needed for coil detachment. Heater elements are typically rigid. If a heater element were to flex, it is likely that adjacent windings would make contact with each other and create a short for the current to bypass the windings. Because heater elements are typically rigid, a shorter heater results in a more flexible pusher tip.

In at least one embodiment the heater is created via 3d printing techniques. Typical heaters utilize coils are formed by winding a single length of wire into a coil. The wire has a resistive quality that generates heat as current passes through the windings. Longer coils offer more resistance and thus generate more heat. As such, the coil must be sufficiently long in order to generate enough heat to sever or melt a connecting member (e.g. a tether) to detach the embolic coil. Using 3d printing techniques, vapor deposition, or additive layer concepts, it would be possible to create a multi-layered heating element that incorporates complex geometries to generate more heat for a given length than a traditional heater coil.

In at least one embodiment, the heater utilizes a plurality of connected planes each including an outer incomplete ring and an inner incomplete ring in the form of a C. The complex shape results in a much longer path for current to travel for a given overall heater length than a traditional coil of a similar length would provide. Thus, much more heat is generated. In some embodiments, a protective shell and dielectric material such as polyimide, parylene, and ceramic can be added to the heater to provide insulation between layers.

One aspect provides an implant delivery system that includes a pusher and a heater capsule at a distal end of the pusher. The heater capsule has an outer capsule and a heater element inside the outer capsule. The heater element defines a plurality of planes, each plane including an inner ring and an outer ring. One of the inner ring and the outer ring of each plane is connected to an inner ring or an outer ring, respectively, of an adjacent plane. The heater element also has an electrical connection to the outer capsule. A first lead wire connects the heater element to a power source and a second lead wire connects the outer capsule to the power source.

The marker band is shaped to allow the coil to pivot around the distal end of the pusher prior to release. This configuration significantly increases the flexibility of the interface between the coil and the delivery system.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is a perspective view of an embodiment of a heater element of the invention;

FIG. 2 is a perspective view of an embodiment of a heater capsule of the invention;

FIG. 3 is a transparent perspective view of an embodiment of a heater capsule of the invention;

FIG. 4 is a transparent plan view of an embodiment of a pusher of the invention;

FIG. 5 is a plan view of an embodiment of a marker band of the invention inserted into a proximal end of an implantable coil;

FIG. 6 is a transparent elevation of an embodiment of a marker band of the invention inserted into a proximal end of an implantable coil; and,

FIG. 7 is a plan view of an embodiment of a marker band of the invention connected to a pusher.

### DETAILED DESCRIPTION OF THE INVENTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

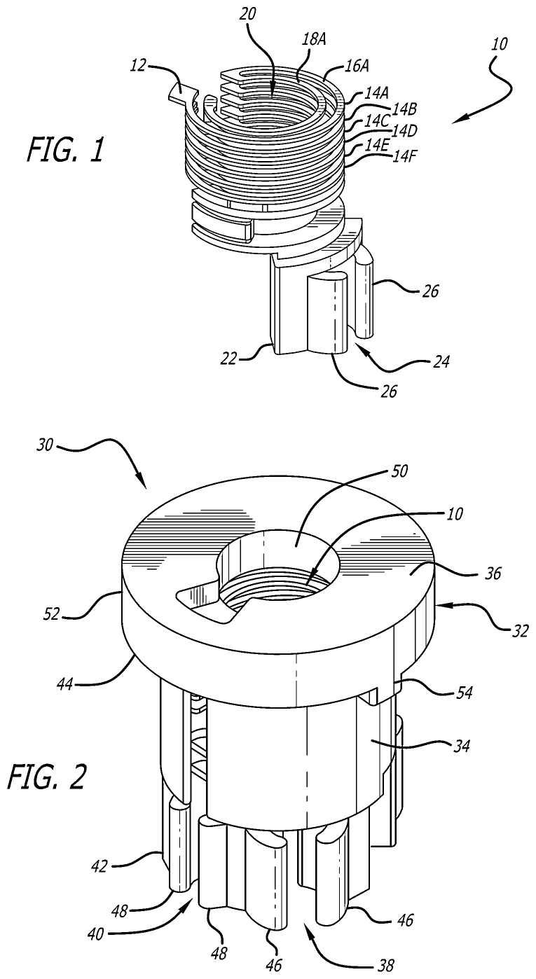

FIG. 1 shows an embodiment of a heater element 10 in accordance with the present invention. The heater element is a complex shape formed through an additive manufacturing process such as 3D printing.

This heater element embodiment 10 is of a unitary construction and includes a complex, double element. The double element is a continuous length of material that begins at a distal tab 12 and extends in a first plane 14A around an outer ring 16A of the C-shape, or incomplete circle shape, and then turns inwardly to form an inner ring 18A of the C-shape, in the same first plane 14A. The inner ring extends back to the other end of the C, adjacent the tab 12, at which point the element plane connects to an adjacent, second plane 14B and forms another C-shaped layer, beginning this time with the inner ring 18B and ending with an outer ring 16B. This pattern is repeated until a number of planes (six planes 14A-F are shown in the example of FIG. 1) are formed sufficient to generate enough heat to break a tether that extends through the heater and is used to connect the implant to the pusher. This repeating pattern with inner and outer rings effectively doubles the amount of element length for a given length of the heater 10. As such, the heating element 10 of the present invention can generate as much heat as a conventional heating element having twice the longitudinal length.

Through additive manufacturing, alternative complex element shapes could be developed that would not be possible with more conventional manufacturing methods. One skilled in the art will realize the present invention should not be construed to be limited to the C-shaped embodiment of FIG. 1. This is just an example of a double-layer, nested element design that generates significant heat over a short longitudinal length. Other examples may include, but are not limited to, planar wave designs, vertical wave designs, triple-nested designs, angled planes, etc.

According to one embodiment, the heater element 10 defines an internal passage 20. The internal passage 20 may be sized to solely contain a tether connecting the implant to the pusher 60. According to another embodiment, the internal passage 20 may be large enough to contain the tether and other components, such as a support mandrel or electrical wires.

A proximal end 22 of the heater element 10 includes a passage 24 defined between tabs 26. The passage 24 is sized to receive a lead wire 68 (FIG. 4). In the example provided, the lead wire 68 is a positive lead wire. The tabs 26 are for use in joining methods such as laser welding, soldering, or the like. In one non-limiting example, the passage 24 between the tabs 26 is sized such that the terminal lead wire 68 has a friction fit between the tabs 26. Thus, the tabs 26 may hold the terminal lead wire 68 in place during a laser welding operation or other joining method. Alternatively, the tabs may be spaced apart wider than the lead wire to allow ease of insertion.

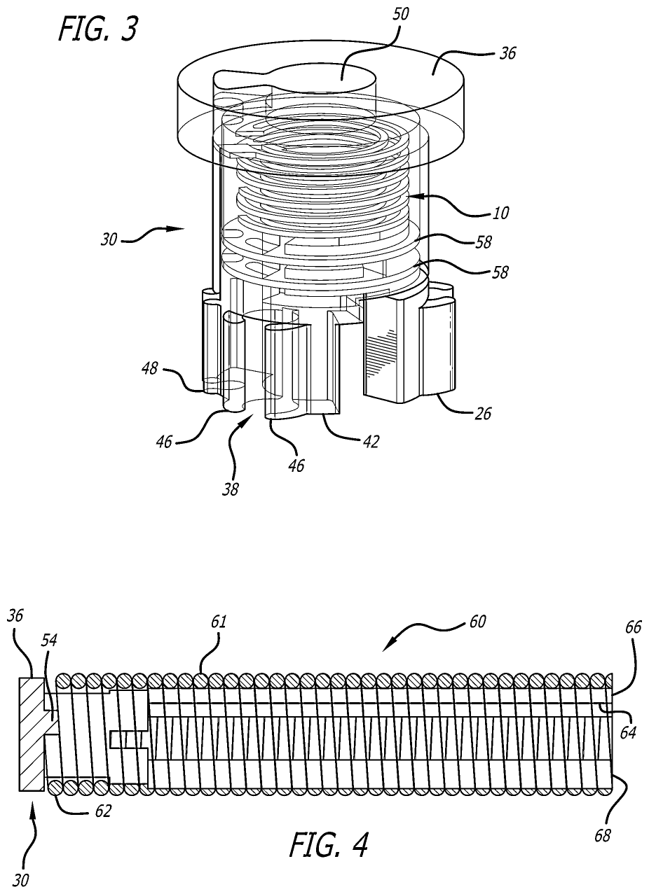

FIGS. 2 and 3 show an embodiment of a heater element 10 as part of a heater capsule 30 of the invention. The heater capsule 30 may be used with a pusher 60 (FIG. 4) usable to deliver an embolic coil to a treatment site, such as an aneurysm. The heater capsule 30 generally includes an outer capsule 32 at least partially surrounding an inner heater element 10. The outer capsule 32 functions to protect the inner heater element 10 and is configured to mate with the distal end 62 of the pusher 60, as shown in FIG. 4. Once installed in the pusher, the heater capsule can be used to release an implant from a pusher, as shown FIG. 20 of U.S. Pat. No. 9,867,622 to Bowman.

In the embodiment of FIG. 2, the outer capsule 32 includes a body 34 and a cap 36. The body 34 has openings 38 and 40 at a proximal end 42 of the body 34, opposite the cap 36, which is at a distal end 44 of the body 34. The openings 38 and 40 are sized to receive wires, such as terminal leads, and include protruding tabs 46 and 48 for use in joining the leads to the tabs through the use of methods such as laser welding, soldering, or the like.

In one embodiment, opening 38 is used to receive a negative terminal lead wire 66 (FIG. 4) used in completing a circuit that provides power to the heater capsule 30. Thus, the outer capsule 32 is constructed of an electrically conductive material such as copper, silver, steel, brass, gold, and alloys thereof, just to name some non-limiting examples. Non-metallic electrically conductive materials may also be used. The opening 38 is defined by the gap between the tabs 46 and is sized to receive the terminal lead wire 66. In one example, the gap between the tabs 46 is sized such that the terminal lead wire 66 has a friction fit between the tabs 46. Thus, the tabs 46 may hold the terminal lead wire 66 in place during the laser welding operation or other joining method.

Opening 40 may be used to attach a stretch-resistant wire 64 (FIG. 2) that extends proximally through the pusher 60. The stretch resistant wire 64 may prevent a coil 61 of the pusher 60 from unduly stretching during movement within a patient. In one example, this stretch-resistant wire 64 is a stainless steel wire which can be welded to the tabs 48 using laser-welding. The stretch resistant wire 64 may also be a polymer tether. The opening 40 is defined by a gap between the tabs 48 and is sized to receive the stretch-resistant wire 64. In one example, the gap between the tabs 48 is sized such that the stretch-resistant wire 64 has a friction fit between the tabs 48 such that the tabs 48 may hold the stretch-resistant wire 64 in place during the laser welding operation or other joining method. Alternatively, the tabs may be spaced apart wider than the lead wire to allow ease of insertion.

The cap 36 of the capsule 32 defines a lumen 50, through which a tether is routed for connecting the implant to the pusher 60. The lumen 50 may have a diameter that matches or approximates the diameter of the internal passage 20 of the heater element 10. The tether passes through the heater 10 and is severed, melted or broken by the heater 10 when the heater 10 is activated in order to release the implant. The cap has a diameter that is greater than the capsule body 34 such that an overhang 52 is created. The overhang 52 acts as a stop against the pusher 60 when the capsule 32 is inserted into the distal end 62 of the pusher coil 61. A protrusion 54 extends proximally from the proximal side of the cap 36 and acts against a distal terminus 62 of the pusher coil 61.

In the example of FIGS. 1-4, the circuit providing power to the heater element 10 begins at a power source in the handle (not shown) at a proximal end of the pusher 60. The positive lead wire 68 extends through the pusher 60 and is electrically connected to the tabs 26 of the heater 10. The current flows through the inner and outer elements 18 and 16, respectively of the various planes 14F-A and up to the distal tab 12, which is electrically connected to the outer capsule 32 of the heater capsule 30. The current then flows through the outer capsule 32 to the tabs 46 and back through the negative lead wire 66 back to the power source. The current could also flow in the reverse direction, as one skilled in the art will understand.

FIG. 3 shows a transparent view of an assembled heater capsule 30. Included in this view are insulative layers 58, which ensure that the current flowing through the heater does not short to the outer capsule 32 prematurely before reaching the distal tab 12. One advantage of the 3d printing techniques used in making the heater is that the insulative layers 58 can be formed concurrently with the heater elements. The insulative layers 58 may be formed of an insulative material such as such as polyimide, parylene, and ceramic, just to name a few non-limiting examples.

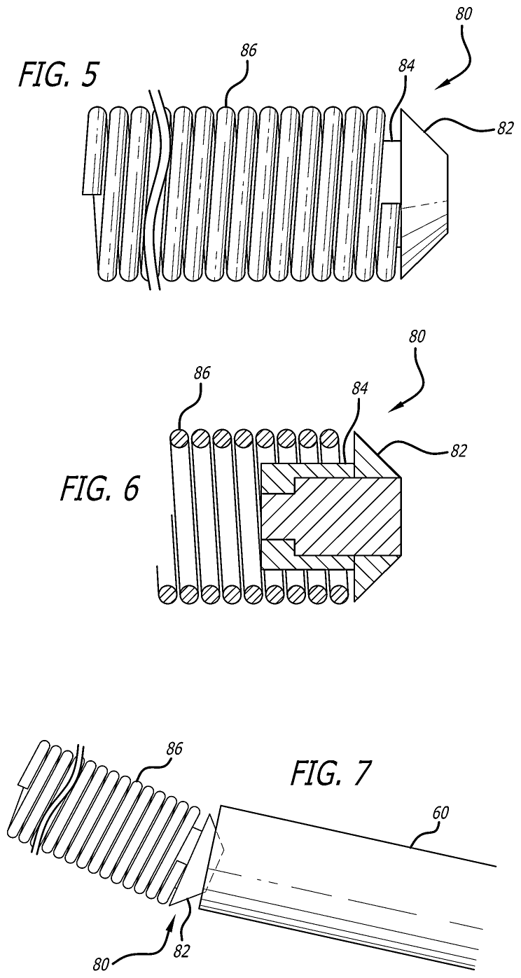

Many delivery pushers used to deliver an embolic coil utilize a marker band just proximal of the embolic coil. The embodiments of FIGS. 5-7 utilize a marker band 80 that includes a proximal portion 82 that is shaped to allow more implant flexibility especially once the implant is outside of the delivery catheter. A distal portion 84 of the marker is smaller and sits within the implant while the proximal portion 82 of the marker is enlarged and has a tapered shape. The distal portion 84 of the marker supports the implant 86 while the implant 86 is connected to the delivery pusher, while the angled marker still allows a bit of freedom of movement as the implant is pushed out from the delivery catheter, thereby reducing the friction and “kick” associated with overly stiff implants during delivery. FIG. 6 is transparent to better show this distal portion 84 within the implant 86. The distal portion 84 may be welded in place.

The example of FIGS. 5-7 is a straight taper, but other embodiments would also accomplish the pivot result. For example, the proximal portion 82 could be rounded, chamfered, beveled, just to name a few non-limiting examples.

FIG. 7 demonstrates how the tapered portion 82 interfaces with the cap 36 of the outer capsule 32 of the heater capsule 30. The proximal portion 82 is tapered to nest within a distal structure at the distal end of the pusher 60. Because the tapered portion 82 has a tapered diameter that is smaller than the diameter of the distal end of the pusher, the marker 80 is allowed to pivot within the distal end of the pusher, much like a ball and socket joint. This allows a greater degree of freedom when pushing the implant coil into the aneurysm.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An implant delivery system comprising:
a pusher;
a heater capsule at a distal end of the pusher and including:
an outer capsule;
a heater element defining a plurality of planes, each plane including an inner ring and an outer ring, wherein one of said inner ring and said outer ring of each plane is connected to an inner ring or an outer ring, respectively, of an adjacent plane, said heater element further having an electrical connection to said outer capsule;
a first lead wire connected to said heater element;
a second lead wire connected to said outer capsule.

2. The implant delivery system of claim 1 wherein said first lead wire comprises a positive lead wire.

3. The implant delivery system of claim 1 further comprising at least one insulative layer disposed between two of said planes.

4. The implant delivery system of claim 1 wherein said heater element defines an internal passage.

5. The implant delivery system of claim 4 wherein said internal passage is sized to receive a tether.

6. The implant delivery system of claim 1 wherein said outer capsule includes tabs used for electrically attaching said second lead wire to said outer capsule.

7. The implant delivery system of claim 1 wherein said heater element includes tabs used for electrically attaching said first lead wire to said heater element.

8. The implant delivery system of claim 1 further comprising a stretch-resistant wire extending through said pusher to said outer capsule.

9. The implant delivery system of claim 8 wherein said outer capsule includes tabs used for attaching said stretch-resistant wire to said outer capsule.

10. The implant delivery system of claim 1, wherein each of the plurality of planes is comprised of a C-shape.

11. The implant delivery system of claim 10, wherein the C-shape is transverse to a longitudinal axis of the heater element.

12. A heater capsule for an implant delivery system, comprising:
a heater element comprising a plurality of C-shaped layers; and
an outer capsule at least partially surrounding the heater element;
wherein each of the plurality of C-shaped layers comprises an outer ring and an inner ring.

13. The heater capsule of claim 12, wherein the plurality of C-shaped layers define a slot extending parallel to a longitudinal axis of the heater element.

14. The heater capsule of claim 12, wherein the plurality of C-shaped layers re each transverse to a longitudinal axis of the heater element.

15. The heater capsule of claim 12, wherein the heater element is electrically connected to the outer capsule.

16. The heater capsule of claim 12, wherein the outer ring of each of the plurality of C-shaped layers is connected to the outer ring of an adjacent C-shaped layer of the plurality of C-shaped layers.

17. The heater capsule of claim 12, wherein the inner ring of each of the plurality of C-shaped layers is connected to the inner ring of an adjacent C-shaped layer of the plurality of C-shaped layers.

18. The heater capsule of claim 12, wherein, for each of the plurality of C-shaped layers, the outer ring is connected to the inner ring.

19. The heater capsule of claim 12, wherein a distal-most outer ring of the plurality of C-shaped layers includes a tab, wherein the tab is electrically connected to the outer capsule.

20. A heater capsule for an implant delivery system, comprising:
a heater means for severing a tether, wherein the heater means is comprised of a plurality of C-shaped layers; and
an outer capsule means for protecting the heater means, wherein the outer capsule means at least partially surrounds the heater means;
wherein each of the plurality of C-shaped layers comprises an outer ring and an inner ring; and
wherein the heater means is electrically connected to the outer capsule.
